(19) United States
(12) Patent Application Publication
(10) Pub. No.: US 2021/0208109 A1
(43) Pub. Date: Jul. 8, 2021

(54) PHOTOACOUSTIC IMAGING DEVICE

(71) Applicant: OLYMPUS CORPORATION, Tokyo (JP)

(72) Inventors: Yoshiaki MURAYAMA, Tokyo (JP); Atsushi DOI, Tokyo (JP); Kentaro IMOTO, Tokyo (JP)

(73) Assignee: OLYMPUS CORPORATION, Tokyo (JP)

(21) Appl. No.: 17/207,897

(22) Filed: Mar. 22, 2021

Related U.S. Application Data
(63) Continuation of application No. PCT/JP2018/035467, filed on Sep. 25, 2018.

(51) Int. Cl. G01N 29/24; G01N 29/28; G01N 29/06

(52) U.S. Cl. CPC G01N 29/2418; G01N 29/0654; G01N 29/28

(57) ABSTRACT

A photoacoustic imaging device includes an irradiator irradiating a specimen with excitation light, a detection unit detecting an acoustic wave at an irradiated position, a propagation body including a bag-shaped body including a film deforming in conformity with a surface shape of the specimen, an interior of the bag-shaped body being filled with an acoustic wave propagation medium, and a processor including hardware and configured to generate an image based on the detected acoustic wave, wherein the propagation body is disposed between the detection unit and the specimen, without interposing any air layers, and moves along with relative movement of the detection unit and the specimen, the detection unit includes an optics collecting the acoustic wave, the propagation body is attached to the optics, and a difference of refractive indexes between the optics and the propagation body is smaller than a difference of refractive indexes between the optics and air.

### CROSS-REFERENCE TO RELATED APPLICATIONS

This is a continuation of International Application PCT/JP2018/035467 which is hereby incorporated by reference herein in its entirety.

### TECHNICAL FIELD

The present invention relates to a photoacoustic imaging device.

### BACKGROUND ART

A photoacoustic imaging device is known in which a specimen is irradiated with pulsed excitation light, and acoustic wave generated in the specimen is detected to thereby acquire an image of the specimen (see PTL 1, for example).

To detect the acoustic wave generated in the specimen without any loss, the device of PTL 1 includes a water tank in which a space between the specimen and an acoustic detector is filled with an acoustic wave propagation medium such as water. The water tank has a form of an open container opened upward, and including, as a bottom surface, a membrane brought into contact closely with an upper surface of the specimen. Furthermore, the acoustic detector is brought into contact closely with water surface of water stored in the container.

### CITATION LIST

### Patent Literature

### PTL 1

Japanese Translation of PCT International Application Publication No. 2011-519281

### SUMMARY OF INVENTION

An aspect of the present invention is a photoacoustic imaging device including an irradiator that irradiates a specimen with excitation light, an acoustic wave detection unit that detects an acoustic wave generated at a position irradiated with the excitation light by the irradiator, an acoustic wave propagation body including a bag-shaped body including a film that deforms in conformity with a surface shape of the specimen, an interior of the bag-shaped body being filled with an acoustic wave propagation medium, and a processor including hardware, the processor being configured to generate an image based on the detected acoustic wave, wherein the acoustic wave propagation body is disposed between the acoustic wave detection unit and the specimen, without interposing any air layers, and moves relative to the specimen along with relative movement of the acoustic wave detection unit and the specimen during image acquisition of the specimen, the acoustic wave detection unit includes an acoustic wave collection optics that collects the acoustic wave, and a converter that converts the acoustic wave collected by the acoustic wave collection optics to an electric signal, the acoustic wave propagation body is attached to the acoustic wave collection optics, and a difference of refractive indexes between the acoustic wave collection optics and the acoustic wave propagation body is smaller than a difference of refractive indexes between the acoustic wave collection optics and air.

Furthermore, another aspect of the present invention is a photoacoustic imaging method including interposing, between a specimen and an acoustic wave detection unit, an acoustic wave propagation body including a bag-shaped body including a film that deforms in conformity with a surface shape, an interior of the bag-shaped body being filled with an acoustic wave propagation medium, without interposing any air layers, irradiating the specimen with excitation light from a light source while moving the light source, the acoustic wave detection unit and the acoustic wave propagation body relative to the specimen, detecting an acoustic wave generated at a position irradiated with the excitation light through the acoustic wave propagation body by the acoustic wave detection unit, and generating an image based on the detected acoustic wave, wherein the acoustic wave detection unit includes an acoustic wave collection optics that collects the acoustic wave, and a converter that converts the acoustic wave collected by the acoustic wave collection optics to an electric signal, the acoustic wave propagation body is attached to the acoustic wave collection optics, and a difference of refractive indexes between the acoustic wave collection optics and the acoustic wave propagation body is smaller than a difference of refractive indexes between the acoustic wave collection optics and air.

### BRIEF DESCRIPTION OF DRAWINGS

FIG. 1 is an entire configuration diagram showing a photoacoustic imaging device according to an embodiment of the present invention.

FIG. 2 is a partially enlarged view showing a state where an acoustic wave propagation body of the photoacoustic imaging device of FIG. 1 is disposed above a specimen.

FIG. 3 is a partially enlarged view showing a state where the specimen is raised from the state of FIG. 2 to bring a surface of the specimen into contact closely with the acoustic wave propagation body.

FIG. 4 is a flowchart explaining a photoacoustic imaging method in which the photoacoustic imaging device of FIG. 1 is used.

FIG. 5 is a partially enlarged view showing a case where a protrusion is present on the surface of the specimen in the photoacoustic imaging device of FIG. 3.

FIG. 6 is a partially enlarged view showing a modification of the photoacoustic imaging device of FIG. 1.

FIG. 7 is a partially enlarged view showing another modification of the photoacoustic imaging device of FIG. 1.

### DESCRIPTION OF EMBODIMENTS

Hereinafter, description will be made as to a photoacoustic imaging device 1 and a photoacoustic imaging method according to an embodiment of the present invention with reference to the drawings.

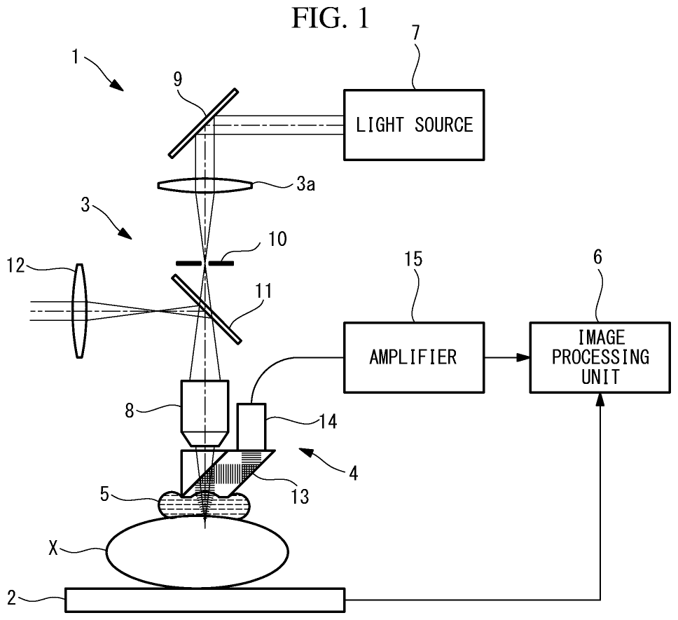

The photoacoustic imaging device 1 according to the present embodiment comprises, as shown in FIG. 1, a stage 2 on which a specimen X is mounted, an excitation light irradiation unit 3 that irradiates the specimen X mounted on the stage 2 with laser light (excitation light), an acoustic wave detection unit 4 that detects an acoustic wave generated in the specimen X irradiated with the laser light, an acoustic wave propagation body 5 attached to the acoustic wave detection unit 4, and an image processing unit (an image generation unit) 6 that generates an image based on the detected acoustic wave. In the drawing, numeral 7 denotes a light source that generates pulsed laser light.

The stage 2 can move the mounted specimen X in a three-dimensional direction. That is, the stage 2 is moved upward and downward in a vertical direction relative to an objective lens 8 described later, so that a focal position of the objective lens 8 can be moved in a depth direction of the specimen X. Furthermore, the stage 2 is moved in a horizontal direction relative to the objective lens 8, so that a position to be irradiated with the laser light can be adjusted in the horizontal direction.

The excitation light irradiation unit 3 includes the objective lens 8 that condenses the pulsed laser light generated in the light source 7, in a region of interest of the specimen X. In the drawing, reference sign 3a denotes a condenser lens, numeral 9 denotes a mirror, numeral 10 denotes a pinhole, numeral 11 denotes a beam splitter, and numeral 12 denotes an eyepiece lens.

The acoustic wave detection unit 4 includes a branch element (an acoustic wave collection optics) 13 that branches the acoustic wave generated in the specimen X from an optical path of the laser light, and an acoustic wave transducer (a converter) 14 disposed in contact with an upper surface of the branch element 13. The acoustic wave transducer 14 outputs intensity of the detected acoustic wave as an electric signal. In the drawing, numeral 15 denotes an amplifier that amplifies the electric signal outputted from the acoustic wave transducer 14.

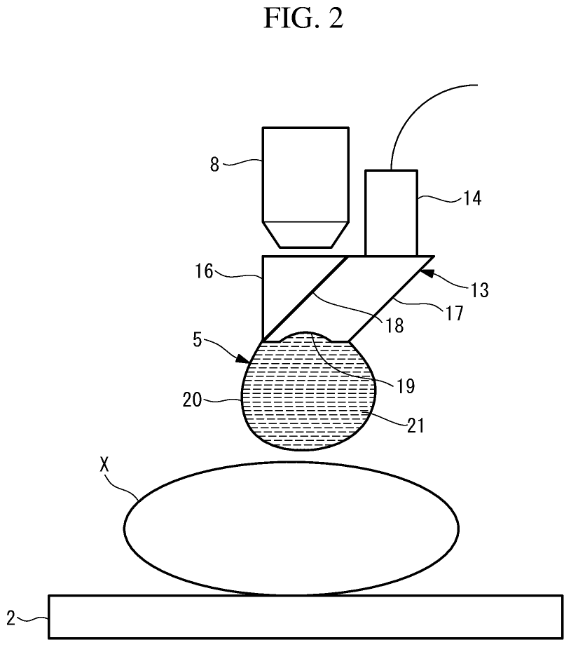

As shown in FIG. 2, the branch element 13 has a configuration of a triangular prism 16 combined with a parallelogram prism 17, and is disposed close to a tip of the objective lens 8.

An inclined surface of the triangular prism 16 and an inclined surface of the parallelogram prism 17 that are arranged adjacent to each other are separated by a liquid disposed between both the surfaces, that is, a nonvolatile liquid with a matched optical refractive index and a low acoustic impedance, such as a thin layer of low molecular weight silicone oil. This layer forms a branch surface 18.

An upper surface of the triangular prism 16 disposed facing and below the tip of objective lens 8 is disposed orthogonally to an optical axis of the objective lens 8.

Consequently, the laser light that exits from the objective lens 8, to enter the triangular prism 16 is transmitted by the branch surface 18 and is emitted from a lower surface of the parallelogram prism 17 to outside the branch element 13. In this case, the laser light is inhibited from being refracted in the upper surface of the triangular prism 16 and the branch surface 18, and the specimen X vertically below the objective lens 8 is straightly irradiated with the laser light emitted from the objective lens.

In the present embodiment, the laser light exits from the lower surface of the parallelogram prism 17, and the acoustic wave enters the lower surface. In this lower surface, a recess (an acoustic lens) 19 that collects the entering acoustic wave is provided. The acoustic wave that enters the branch element 13 from the lower surface of the parallelogram prism 17 is collected in the recess 19 to enter the parallelogram prism 17, reflected by the branch surface 18 and a facing surface parallel to the branch surface 18, in the parallelogram prism 17, and then exits from an upper surface of the parallelogram prism 17 adjacent to the facing surface to outside the branch element 13. On this upper surface, the acoustic wave transducer 14 is disposed, so that the acoustic wave can be detected.

The acoustic wave propagation body 5 is formed of a bag-shaped body 20 having a thin film, an interior of the bag-shaped body being filled with an acoustic wave propagation medium 21 having a refractive index equal to that of the branch element 13 and the bag-shaped body 20, such as water. The bag-shaped body 20 is made of a material that can transmit the laser light and acoustic wave, such as silicone rubber, and has properties of deforming in conformity with a shape of an object in contact, and coming into contact closely with the object without any gaps. Furthermore, the bag-shaped body 20 entirely including the film is illustrated, but the present invention is not limited to this example, and a bag-shaped body in which a part that is not in contact with the object is not a film, may be adopted.

The bag-shaped body 20 is filled with the acoustic wave propagation medium 21 without any air layers formed. The bag-shaped body 20 is attached to the lower surface of the parallelogram prism 17 that is an exit surface of the laser light and an entrance surface of the acoustic wave, and the acoustic wave propagation medium 21 is embedded in the recess 19. The acoustic wave propagation medium 21 having a refractive index equal to that of the parallelogram prism 17 is embedded in the recess 19, and hence, the recess does not have any condensing action to the laser light, and performs a lens function only to the acoustic wave.

The image processing unit 6 generates the image based on the signal amplified by the amplifier 15 and positional information of the stage 2.

Description will be made as to a photoacoustic imaging method in which the photoacoustic imaging device 1 according to the present embodiment including such a configuration as described above is used, with reference to the drawings.

To perform observation of the specimen X by use of the photoacoustic imaging device 1 according to the present embodiment, the specimen X, such as a mouse, is mounted on the stage 2, and as shown in FIG. 2, the stage 2 is raised from a state where the specimen X is disposed vertically below the acoustic wave propagation body 5, thereby bringing the acoustic wave propagation body 5 into contact with an upper surface of the specimen X.

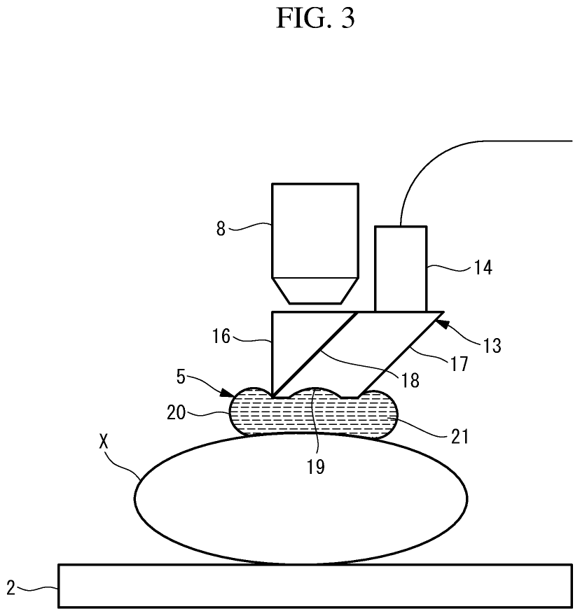

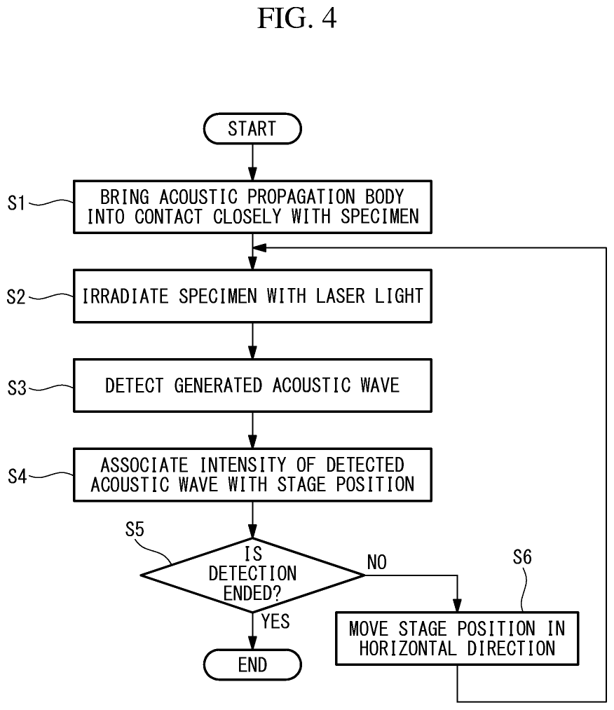

If the stage 2 is further raised in this state, as shown in FIG. 3 and FIG. 4, the acoustic wave propagation body 5 deforms in conformity with a surface shape of the specimen X to come into contact closely with the surface of the specimen X without any gaps (step S1). Then, when the stage 2 is raised to a position where the focal position of the objective lens 8 is disposed at a desirable position in the specimen X, the pulsed laser light is generated from the light source 7. Then, the laser light passes through the condenser lens 3a, the pinhole 10 and the beam splitter 11, to be condensed by the objective lens 8, and the laser light is transmitted by the branch element 13 and the acoustic wave propagation body 5, to enter the specimen X (step S2).

The branch element 13 and the acoustic wave propagation body 5 have an equal refractive index, and the laser light from the objective lens 8 enters the specimen in a direction orthogonal to the surface of the branch element 13. Therefore, the laser light travels straight without being refracted, and is focused in the specimen X.

The laser light that enters the specimen X generates the acoustic wave at the focal position of the objective lens 8. When a part of the generated acoustic wave that returns to an acoustic wave propagation body 5 side is transmitted by the acoustic wave propagation body 5 to enter the branch element 13, the part is collected by the recess 19. Then, the acoustic wave is reflected by the branch surface 18 and the facing surface in the parallelogram prism 17, and detected by the acoustic wave transducer 14 disposed in contact with the parallelogram prism 17 (step S3). The acoustic wave propagation body 5 is in close contact with the surface of the specimen X, and the interior of the bag-shaped body 20 of the acoustic wave propagation body 5 is filled with the acoustic wave propagation medium 21. Therefore, any air layers are not present between the specimen X and the acoustic wave transducer 14, and the acoustic wave can be detected while reducing attenuation of the acoustic wave.

The detected acoustic wave is amplified by the amplifier 15, and is then associated with the positional information of the stage 2, in the image processing unit 6 (step S4). Afterward, it is determined whether or not the acoustic wave is detected at all irradiated positions (step S5). In a case where the detection is not ended, the stage 2 is moved by a predetermined distance in the horizontal direction, to thereby change the irradiated position with the laser light in the horizontal direction (step S6). The steps are repeated from the step S2, so that an image indicating an intensity distribution of the acoustic wave can be generated in the image processing unit 6.

In this case, if the stage 2 is moved, the surface shape of the specimen X in contact with the acoustic wave propagation body 5 changes. However, the acoustic wave propagation body 5 deforms in conformity with the surface shape of the specimen X every time, and a close contact state of the propagation body with the surface of the specimen X is maintained. Consequently, even when the irradiated position with the laser light is changed, the acoustic wave can be detected while reducing the attenuation.

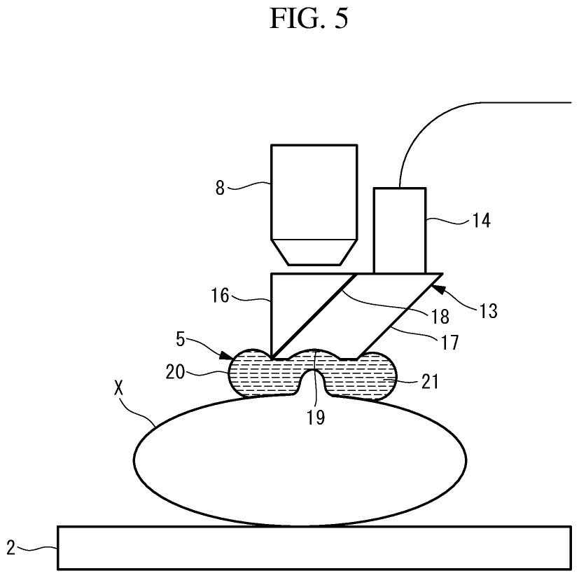

In particular, as shown in FIG. 5, even in a case where unevenness due to the protrusion or the like is present on the surface of the specimen X, the shape of the acoustic wave propagation body 5 is changed in conformity with the surface shape, and hence, the acoustic wave can continue to be stably detected. For example, even when the acoustic wave propagation medium 21 in a form of gel is applied to the surface of the specimen X, imaging can be performed in correspondence to a certain degree of unevenness. According to the present embodiment, however, imaging can be easily performed even in correspondence to such unevenness that cannot be coped under surface tension of the gel-like acoustic wave propagation medium 21.

Furthermore, according to the photoacoustic imaging device 1 of the present embodiment, differently from a conventional technology to move the objective lens 8 in a water tank, the acoustic wave propagation body 5 is attached to the acoustic wave detection unit 4. Therefore, it is not necessary to provide a large water tank that covers a relative movement range of the specimen X and the objective lens 8, and there is an advantage that the device can be reduced in size while securing a required imaging range.

Additionally, differently from the water tank in which water surface is formed, the acoustic wave propagation medium 21 is enclosed in the bag-shaped body 20. Consequently, there is also an advantage that generation of a disadvantage such as water scattering due to evaporation of water, mixing of dust and movement of the objective lens 8 can be prevented in advance.

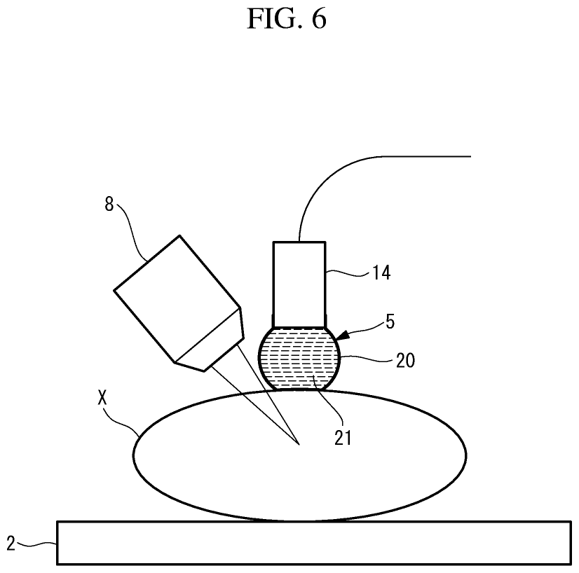

Note that in the present embodiment, the branch element 13 is disposed between the objective lens 8 and the specimen X, and the acoustic wave propagation body 5 is attached to the branch element 13. Alternatively, as shown in FIG. 6, laser light may enter a specimen X without passing through an acoustic wave propagation body 5, and the acoustic wave propagation body 5 may be attached directly to an acoustic wave transducer 14.

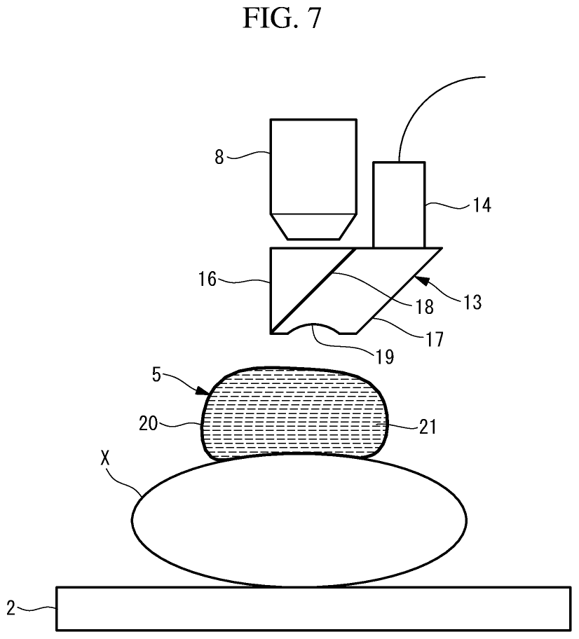

Furthermore, in the present embodiment, the acoustic wave propagation body 5 including the bag-shaped body 20 in which the acoustic wave propagation medium 21 is enclosed is attached to the acoustic wave detection unit 4. Alternatively, as shown in FIG. 7, an independent acoustic wave propagation body 5 may be interposed between an acoustic wave detection unit 4 and the specimen X.

Additionally, in the present embodiment, the stage 2 on which the specimen X is mounted is moved in a three-dimensional direction, to move the specimen X relative to the acoustic wave detection unit 4. Alternatively, the stage 2 may be fixed, and the acoustic wave detection unit 4 may be moved in a three-dimensional direction.

Water is illustrated as the acoustic wave propagation medium 21, but any other acoustic wave propagation medium 21 may be adopted.

The above-described embodiment also leads to the following aspects.

An aspect of the present invention is a photoacoustic imaging device including an excitation light irradiation unit that irradiates a specimen with excitation light, an acoustic wave detection unit that detects an acoustic wave generated at a position irradiated with the excitation light by the excitation light irradiation unit, an acoustic wave propagation body including a bag-shaped body including a film that deforms in conformity with a surface shape of the specimen, an interior of the bag-shaped body being filled with an acoustic wave propagation medium, and an image generation unit that generates an image based on the detected acoustic wave, wherein the acoustic wave propagation body is disposed between the acoustic wave detection unit and the specimen, without interposing any air layers, and moves relative to the specimen along with relative movement of the acoustic wave detection unit and the specimen during image acquisition of the specimen.

According to the present aspect, upon the irradiation of the specimen with pulsed excitation light, the acoustic wave is generated at the irradiated position of the specimen, and detected via the acoustic wave propagation body in close contact with the specimen and the acoustic wave detection unit, by the acoustic wave detection unit. In the image generation unit, it is possible to measure and arrange a series of amplitudes of the acoustic wave at positions of the specimen to generate an acoustic wave image of the specimen.

In this case, the acoustic wave propagation body moves relative to the specimens along with the relative movement of the acoustic wave detection unit and the specimen. Therefore, even though the acoustic wave propagation body is smaller than an imaging range, the film that forms the bag-shaped body is deformed in conformity with the surface shape of the specimen, at each position of a required imaging range, to fill a space between the acoustic wave detection unit and the specimen. An interposed air layer can therefore be eliminated and loss in acoustic wave can be suppressed. Consequently, the device can be reduced in size while securing the required imaging range, without preparing any large water tanks.

In the above aspect, the acoustic wave propagation body may be attached to the acoustic wave detection unit.

According to this configuration, it is assured that the acoustic wave propagation body can move relative to the specimen along with the relative movement of the acoustic wave detection unit and the specimen.

Furthermore, in the above aspect, the acoustic wave detection unit may include an acoustic wave collection optics that collects the acoustic wave, and a converter that converts the acoustic wave collected by the acoustic wave collection optics to an electric signal, and the acoustic wave propagation body may be attached to the acoustic wave collection optics.

According to this configuration, the acoustic wave generated in the specimen is collected through the acoustic wave propagation body by the acoustic wave collection optics, and converted to the electric signal by the converter.

Additionally, in the above aspect, the acoustic wave detection unit may include a converter that converts the acoustic wave to an electric signal, and the acoustic wave propagation body may be attached to the converter.

According to this configuration, the acoustic wave generated in the specimen directly enters the converter through the acoustic wave propagation body, and is converted to the electric signal in the converter.

Furthermore, another aspect of the present invention is a photoacoustic imaging method including interposing, between a specimen and an acoustic wave detection unit, an acoustic wave propagation body including a bag-shaped body including a film that deforms in conformity with a surface shape, an interior of the bag-shaped body being filled with an acoustic wave propagation medium, without interposing any air layers, irradiating the specimen with excitation light from a light source while moving the light source, the acoustic wave detection unit and the acoustic wave propagation body relative to the specimen, detecting an acoustic wave generated at a position irradiated with the excitation light through the acoustic wave propagation body by the acoustic wave detection unit, and generating an image based on the detected acoustic wave.

### REFERENCE SIGNS LIST

1 photoacoustic imaging device
3 excitation light irradiation unit
4 acoustic wave detection unit
5 acoustic wave propagation body
6 image processing unit (an image generation unit)
7 light source
13 branch element (an acoustic wave collection optics)
14 acoustic wave transducer (a converter)
20 bag-shaped body
21 acoustic wave propagation medium
X specimen

1. A photoacoustic imaging device comprising:
an irradiator that irradiates a specimen with excitation light,
an acoustic wave detection unit that detects an acoustic wave generated at a position irradiated with the excitation light by the irradiator,
an acoustic wave propagation body comprising a bag-shaped body including a film that deforms in conformity with a surface shape of the specimen, an interior of the bag-shaped body being filled with an acoustic wave propagation medium, and
a processor comprising hardware, the processor being configured to generate an image based on the detected acoustic wave,
wherein the acoustic wave propagation body is disposed between the acoustic wave detection unit and the specimen, without interposing any air layers, and moves relative to the specimen along with relative movement of the acoustic wave detection unit and the specimen during image acquisition of the specimen,
the acoustic wave detection unit comprises:
an acoustic wave collection optics that collects the acoustic wave; and
a converter that converts the acoustic wave collected by the acoustic wave collection optics to an electric signal,
the acoustic wave propagation body is attached to the acoustic wave collection optics, and
a difference of refractive indexes between the acoustic wave collection optics and the acoustic wave propagation body is smaller than a difference of refractive indexes between the acoustic wave collection optics and air.

2. The photoacoustic imaging device according to claim 1, wherein the acoustic wave propagation medium comprises silicone rubber having a refractive index equal to that of the acoustic wave collection optics.

3. The photoacoustic imaging device according to claim 1, wherein the interior of the bag-shaped body is filled with the acoustic wave propagation medium without any air layers formed.

4. The photoacoustic imaging device according to claim 1, wherein the acoustic wave collection optics comprises a prism an exit surface of which is a recessed surface, and
the bag-shaped body comes into contact closely with the prism.

5. A photoacoustic imaging method comprising:
interposing, between a specimen and an acoustic wave detection unit, an acoustic wave propagation body comprising a bag-shaped body including a film that deforms in conformity with a surface shape, an interior of the bag-shaped body being filled with an acoustic wave propagation medium, without interposing any air layers,
irradiating the specimen with excitation light from a light source while moving the light source, the acoustic wave detection unit and the acoustic wave propagation body relative to the specimen,
detecting an acoustic wave generated at a position irradiated with the excitation light through the acoustic wave propagation body by the acoustic wave detection unit, and
generating an image based on the detected acoustic wave, wherein
the acoustic wave detection unit comprises:
an acoustic wave collection optics that collects the acoustic wave; and
a converter that converts the acoustic wave collected by the acoustic wave collection optics to an electric signal,
the acoustic wave propagation body is attached to the acoustic wave collection optics, and
a difference of refractive indexes between the acoustic wave collection optics and the acoustic wave propagation body is smaller than a difference of refractive indexes between the acoustic wave collection optics and air.
